(19) United States
(12) Patent Application Publication
(10) Pub. No.: US 2008/0228091 A1
(43) Pub. Date: Sep. 18, 2008

(54) METHOD AND SYSTEM FOR PATIENT EVALUATION

(75) Inventors: Willi Kaiser, Baden (DD); Rolf Band, Gottenheim (DE)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/684,813

(22) Filed: Mar. 12, 2007

(51) Int. Cl. A61B 5/0402

(52) U.S. Cl. 600/509

(57) ABSTRACT

A method for evaluating a patient is disclosed herein. The method includes performing an exercise test on a patient, obtaining an electrocardiogram during the exercise test, converting the electrocardiogram to an audio file, and analyzing the audio file in order to evaluate the patient. A corresponding system for evaluating a patient is also provided.

### FIELD OF THE INVENTION

This disclosure relates generally to a method and system for evaluating a patient.

### BACKGROUND OF THE INVENTION

An electrocardiograph is a cardiac diagnostic/monitoring system adapted to record the electrical activity of a patient's heart. The electrocardiograph generally includes an array of sensors or transducers placed at predetermined positions on a patient's body. An electrocardiograph is commonly implemented during exercise tests wherein a patient is evaluated while undergoing some form of strenuous physical activity such as, for example, running on a treadmill.

The recorded data from an electrocardiograph is generally displayed in the form of a graph that is often referred to as an electrocardiogram (ECG). It is well known that the visual analysis of the various waves that make up an ECG can yield important diagnostic information. One problem is that a visual analysis may not reveal all the information contained in an ECG. Another problem is that a visual analysis may not be the most efficient means for obtaining certain types of ECG information.

### BRIEF DESCRIPTION OF THE INVENTION

The above-mentioned shortcomings, disadvantages and problems are addressed herein which will be understood by reading and understanding the following specification.

In an embodiment, a method for evaluating a patient includes performing an exercise test on a patient, obtaining an electrocardiogram during the exercise test, converting the electrocardiogram to an audio file, and analyzing the audio file in order to evaluate the patient.

In another embodiment, a method for evaluating a patient includes performing an exercise test on a patient, wherein the exercise test comprises an exercise phase and a recovery phase. The method for evaluating a patient also includes obtaining an electrocardiogram during the exercise phase and the recovery phase of the exercise test, converting the electrocardiogram to an audio file, playing the audio file with an audio player, and listening to the audio file in order to evaluate the patient.

In another embodiment, a system for evaluating a patient includes an electrocardiograph configured to record the electrical activity of a patient undergoing an exercise test, and to convey the recorded electrical activity in the form of an electrocardiogram. The system also includes a computer operatively connected to the electrocardiograph. The computer is configured to receive the electrocardiogram from the electrocardiograph and to convert the electrocardiogram into an audio file. The system also includes an audio player configured to play the audio file such that the audio file can be analyzed in order to evaluate the patient.

Various other features, objects, and advantages of the invention will be made apparent to those skilled in the art from the accompanying drawings and detailed description thereof.

### BRIEF DESCRIPTION OF THE DRAWINGS

FIG. 1 is a schematic illustration of a cardiac diagnostic/monitoring system operatively connected to a patient via a twelve lead system in accordance with an embodiment;

FIG. 2 is an electrocardiogram; and

FIG. 3 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

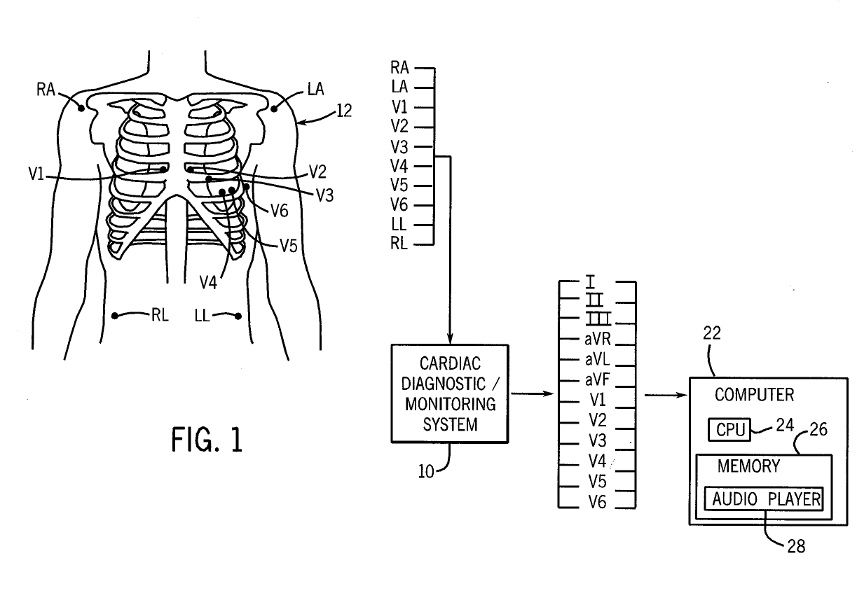

Referring to FIG. 1, a schematically represented cardiac diagnostic/monitoring system 10 is shown. The cardiac diagnostic/monitoring system 10 may, for example, comprise an electrocardiograph. The cardiac diagnostic/monitoring system 10 is adapted measure an electrical signal generated by a patient's heart.

The cardiac diagnostic/monitoring system 10 can be coupled to the patient 12 by an array of sensors or transducers. In the illustrated embodiment, the array of sensors include a right arm electrode RA; a left arm electrode LA; chest electrodes V1, V2, V3; V4, V5 and V6; a right leg electrode RL; and a left electrode leg LL for acquiring a standard twelve lead, ten-electrode electrocardiogram (ECG) signal. The twelve ECG leads include leads I, II, V1, V2, V3, V4, V5 and V6 which are acquired directly from the patient leads, and leads III, aVR, aVL and aVF which are derived using Einthoven's law. In other embodiments, alternative configurations of sensors and sensor locations can be used to acquire a standard or non-standard ECG signal. For example, an expanded fifteen lead system, including four extra electrodes, can be used to form Frank X, Y and Z leads.

The cardiac diagnostic/monitoring system 10 may be operatively connected to a computer 22 so that ECG data from the diagnostic/monitoring system 10 is transferable to the computer 22. The computer 22 includes a central processing unit (CPU) 24 and a storage or memory device 26. The memory device 26 may contain an audio player 28 such as, for example, windows media player. As will be described in detail hereinafter, the CPU 24 is configured to convert the ECG data in to an audio file that can be stored in the memory device 26 and/or played via the audio player 28.

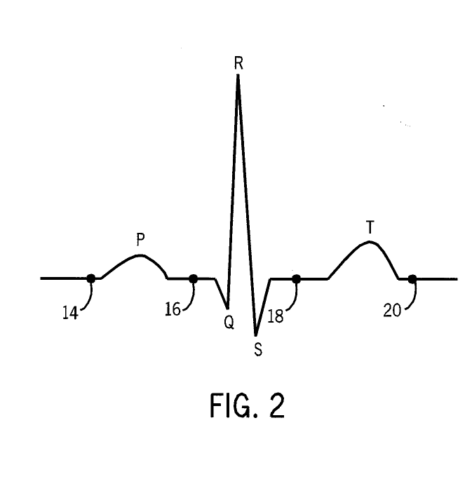

Referring to FIG. 2, an electrocardiogram of a single heartbeat typically referred to as a PQRST complex is shown. The portion of the PQRST complex defined between reference points 14 and 16 is defined as the P-wave, and corresponds to activity in the atria. The portion of the PQRST complex defined between reference points 16 and 18 is defined as the QRS complex, and represents the electrical activation of the ventricles. The portion of the PQRST complex defined between reference points 18 and 20 is defined as the T-wave, and represents the electrical recovery or recharge phase of the ventricles.

It is well known to analyze an ECG signal by visually evaluating the constituent PQRST complex segments. It has been observed, however, that a visual analysis may not reveal all the information contained in the ECG and that a visual analysis may not be the most efficient means for obtaining certain types of ECG information. Therefore, as will be described in detail hereinafter, the ECG can be converted into an audio file, and an auditory evaluation of the converted audio file can be performed for purposes such as patient risk prediction and early disease detection. It should be appreciated that the auditory evaluation of the audio file may be performed independently or in combination with a more conventional visual ECG evaluation.

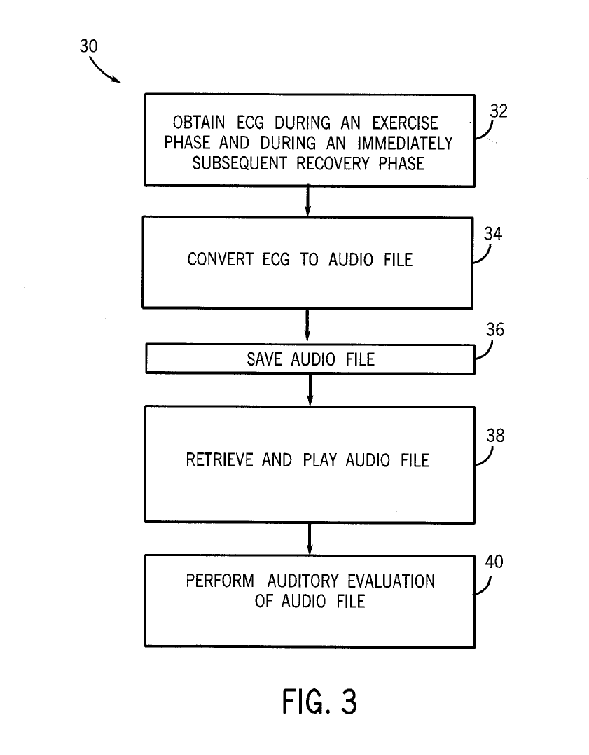

Referring to FIG. 3, a block diagram illustrates a method 30 in accordance with an embodiment. The individual blocks shown in FIG. 2 represent steps that may be performed in accordance with the method 30. Unless otherwise specified, steps 32-40 of the method 30 need not be performed in the order shown.

At step 32, the cardiac diagnostic/monitoring system 10 is coupled to the patient 12 (shown in FIG. 1) and an ECG is obtained during an exercise phase and during an immediately subsequent recovery phase. The “exercise phase” is a period wherein the patient 12 is undergoing some form of physical activity such as, for example, running on a treadmill, and the “recovery phases” is a period wherein the patient is relaxing. The ECG may be obtained in any known manner such as, for example, with the ten-electrode array described hereinabove. It has been observed that ECG data obtained during an exercise test includes a high density of information that is potentially relevant to patient risk prediction and early disease detection. Therefore, ECG data obtained during an exercise test and converted to an audio file can be assessed in a relatively short amount of time (e.g., in several seconds).

The exercise phase of step 32 will hereinafter be described in accordance with an illustrative embodiment wherein the patient 12 (shown in FIG. 1) runs on a treadmill and is subjected to a series of events that increase the patient's requisite activity level in a generally stepwise manner. These events may, for example, include an increase in treadmill speed and/or incline angle.

At step 34, the ECG obtained from step 32 is converted to an audio file such as, for example, a “.wav file” or a “.mp3 file”. This step may be performed using the computer 22 (shown in FIG. 1). The conversion of an electronic file such as an ECG, from one format to another is known to those skilled in the art and therefore will not be described in detail. At step 36, the audio file of step 34 is stored or saved on a conventional storage medium such as the memory device 26 (shown in FIG. 1) so that the audio file can be evaluated at a later time.

At step 38, the audio file is retrieved from storage and played using a known audio player such as the audio player 28 (shown in FIG. 1). The audio file may, according to one embodiment, have a reproduction factor of either 60 or 120. For purposes of this disclosure, a “reproduction factor” refers to the differential between the audio file playback time and the time during which the recorded events actually took place. Therefore, a reproduction factor of 60 means that a reproduction of a 10-minute exercise test can be heard in approximately 10 seconds. Advantageously, this allows a listener to evaluate more information in a shorter amount of time and thereby more quickly assess the patient 12. According to another embodiment, the audio file can be played back in stereo such that the left channel represents the lead V2 (shown in FIG. 1) and the right channel represents the lead V5 (shown in FIG. 1). According to yet another embodiment, the audio file can be played back in stereo such that the left channel represents the lead aVF (shown in FIG. 1) and the right channel represents the lead V2.

At step 40, the audio file is analyzed. This analysis may include an auditory evaluation of the audio file performed by an experienced professional healthcare provider such as a physician. After listening to a large number of audio files corresponding to a plurality of different patients, the healthcare provider will become trained at identifying a wide variety of audible abnormalities that are relevant to patient risk prediction and/or early disease detection. Therefore, the audio file analysis of step 40 should be read to include, in a non-limiting manner, listening to an ECG for purposes of identifying any audible event that may be associated with increased patient risk and/or the presence of a disease.

The following section will provide non-limiting illustrative examples of “normal” auditory evaluations, “abnormal” auditory evaluations, and “borderline” auditory evaluations. For purposes of this disclosure, a “normal” auditory evaluation is one wherein the evaluation does not indicate an increased patient risk or the presence of a disease. An “abnormal” auditory evaluation is one wherein the evaluation strongly indicates an increased patient risk or the presence of a disease, and a “borderline” auditory evaluation is one wherein there is a somewhat weaker indication of increased patient risk or the presence of a disease.

According to an illustrative embodiment, a normal auditory evaluation is one wherein the patient's ECG includes the following audibly detectable characteristics. A first audibly detectable characteristic of a normal evaluation is a generally stepwise increase in heart rate wherein each heart rate increase corresponds to an increase in requisite activity level induced by the exercise test. As previously indicated, the increase in requisite activity level may, for example, be induced by increasing the speed or incline angle of a treadmill. Another audibly detectable characteristic of a normal evaluation is a heart rate that is generally proportional to activity level. In other words, the patient's heart rate steadily increases during the exercise phase and the patient's heart rate steadily decreases during the recovery phase of the exercise test. An increase or decrease in the patient's heart rate is audibly detectable as a corresponding increase or decrease in the frequency of the sound wave representing the patient's ECG.

According to an illustrative embodiment, an abnormal auditory evaluation is one wherein the patient's ECG includes the following audibly detectable characteristics. A first audibly detectable characteristic of an abnormal evaluation is a generally linear increase of heart rate in response to a generally stepwise increase in activity level. This characteristic is abnormal in that the patient's heart rate does not directly respond to an increase in activity level. Another audibly detectable characteristic of an abnormal evaluation is a heart rate that does not steadily decreases during the recovery phase of the exercise test. Some additional audibly detectable conditions indicative of an abnormal auditory evaluation will hereinafter be individually described.

A ventricular premature beat (VPB) or extrasystole is an audibly detectable condition consistent with an abnormal auditory evaluation. As is known to those skilled in the art, a VPB is a form of irregular heartbeat in which the ventricle contracts prematurely. During an exercise test, the beat-to-beat intervals should steadily decrease during the exercise phase and steadily increase during the recovery phase. A VPB interrupts this behavior with a short beat-to-beat interval followed by a long beat-to-beat interval (a compensatory pause). This sequence of events is clearly audible. A cumulative appearance of VPBs in the recovery phase is an indicator for an increased risk of mortality.

T-wave alternans (TWA) refers to a condition wherein there are alternating variations in shape of consecutive T-waves. As previously indicated, the T-wave is the portion of the PQRST complex defined between reference points 18 and 20 (shown in FIG. 2). During TWA, the ECG generally comprises a plurality of even numbered T-waves having a first generally common shape, and a plurality of odd numbered T-waves having a second generally common shape wherein the first shape is distinct from the second shape. The alternating variations in the shape of consecutive T-waves, which are indicative of TWA, are audibly detectable as an additional deeper tone.

Atrial fibrillation (AF) is an abnormal heart rhythm. Heart beats in a normal heart begin after electricity generated in the atria by the sinoatrial node spreads through the heart and causes contraction of the heart muscle and pumping of blood. In AF, the regular electrical impulses of the sinoatrial node are replaced by disorganized, rapid electrical impulses that result in irregular heart beats. AF is audibly detectable as a blurring noise caused by inconsistent or irregular durations between consecutive heart beats.

According to an illustrative embodiment, a borderline auditory evaluation is one wherein the patient's ECG includes VPBs during the exercise phase of the exercise test.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A method for evaluating a patient comprising:
performing an exercise test on a patient;
obtaining an electrocardiogram during the exercise test;
converting the electrocardiogram to an audio file; and
analyzing the audio file in order to evaluate the patient.

2. The method of claim 1, wherein said obtaining an electrocardiogram includes obtaining a twelve lead, ten-electrode electrocardiogram.

3. The method of claim 1, wherein said analyzing the audio file includes listening to the audio file.

4. The method of claim 3, wherein said analyzing the audio file includes playing the audio file with an audio player.

5. The method of claim 4, wherein said playing the audio file with an audio player includes playing the audio file with a reproduction factor of 60 or with a reproduction factor of 120.

6. The method of claim 4, wherein said playing the audio file with an audio player includes playing the audio file in stereo.

7. The method of claim 6, wherein said playing the audio file in stereo includes playing the audio file such that a first stereo channel represents a first electrocardiogram lead and a second stereo channel represents a second electrocardiogram lead.

8. The method of claim 6, wherein said playing the audio file in stereo includes playing the audio file such that a first stereo channel represents an electrocardiogram lead V2 and a second stereo channel represents an electrocardiogram lead V5.

9. The method of claim 6, wherein said playing the audio file in stereo includes playing the audio file such that a first stereo channel represents an electrocardiogram lead aVF and a second stereo channel represents an electrocardiogram lead V2.

10. A method for evaluating a patient comprising:
performing an exercise test on a patient, said exercise test comprising an exercise phase and a recovery phase;
obtaining an electrocardiogram during the exercise phase and the recovery phase of the exercise test;
converting the electrocardiogram to an audio file;
playing the audio file with an audio player; and
listening to the audio file in order to evaluate the patient.

11. The method of claim 10, wherein said obtaining an electrocardiogram includes obtaining a twelve lead, ten-electrode electrocardiogram.

12. The method of claim 11, wherein said playing the audio file with an audio player includes playing the audio file with a reproduction factor of 60 or with a reproduction factor of 120.

13. The method of claim 10, wherein said playing the audio file with an audio player includes playing the audio file in stereo.

14. The method of claim 13, wherein said playing the audio file in stereo includes playing the audio file such that a first stereo channel represents a first electrocardiogram lead and a second stereo channel represents a second electrocardiogram lead.

15. The method of claim 13, wherein said playing the audio file in stereo includes playing the audio file such that a first stereo channel represents an electrocardiogram lead V2 and a second stereo channel represents an electrocardiogram lead V5.

16. A system for evaluating a patient comprising:
an electrocardiograph configured to record the electrical activity of a patient undergoing an exercise test, and to convey the recorded electrical activity in the form of an electrocardiogram;
a computer operatively connected to the electrocardiograph, said computer configured to receive the electrocardiogram from the electrocardiograph and to convert the electrocardiogram into an audio file; and
an audio player configured to play the audio file such that the audio file can be analyzed in order to evaluate the patient.

17. The system of claim 16, wherein the computer includes a memory device configured to store the audio player.

18. The system of claim 16, wherein the audio player is configured to play the audio file with a reproduction factor of 60 or with a reproduction factor of 120.

19. The system of claim 16, wherein the audio player is configured to play the audio file in stereo.

20. The system of claim 19, wherein the audio player is configured to play the audio file such that a first stereo channel represents a first electrocardiogram lead and a second stereo channel represents a second electrocardiogram lead.
